(19) United States
(12) Patent Application Publication
(10) Pub. No.: US 2004/0203005 A1
(43) Pub. Date: Oct. 14, 2004

(54) Dual hybridization of complex nucleic acid samples for sequencing and single-nucleotide polymorphism identification

(76) Inventor: Wanda L. B. White, Oak Ridge, TN (US)

Correspondence Address: BIRCH STEWART KOLASCH & BIRCH, PO BOX 747, FALLS CHURCH, VA, 22040-0747, US

(21) Appl. No.: 10/412,240

(22) Filed: Apr. 14, 2003

(51) Int. Cl. C12Q 1/68

(52) U.S. Cl. 435/6

(57) ABSTRACT

The present invention is drawn to a method of nucleic acid sequencing by hybridizing a selection probe with a conserved region of a target nucleic acid molecule that contains a sequence area of interest; hybridizing a sequencing probe that is different from the selection probe to a variable region of the target nucleic acid molecule; determining the nucleic acid sequence of the area of interest only. The present invention is for the identification and analysis of single nucleotide polymorphisms (SNP).

### FIELD OF THE INVENTION

This invention is related to the fields of deoxyribonucleic acid (DNA) sequencing, ribonucleic acid (RNA) sequencing, genetic diagnostics by hybridization to known nucleic acid and nucleic acid analog sequences, and genetic mapping. The invention is also related to the field of single nucleotide polymorphism (SNP) identification for genetic mapping or genetic diagnostics. The invention is related to the fields of parallel sequencing and single-nucleotide polymorphism identification in nucleic acids.

### BACKGROUND ART

Single Nucleotide Polymorphisms—A SNP is defined as a variation in a DNA sequence in which alleles are defined by a single or a few base changes that occur at a frequency of greater than 1 percent in the population (Mir, K.; & Southern, E.; Sequence Variation in Genes and Genomic DNA: Methods for Large-Scale Analysis in Annual Review of Genomics and Human Genetics, E. Lander, D. Page, and R. Lifton, eds., 2000, 1, pp. 341-355). Single nucleotide polymorphisms (SNPs) are the most frequent form of DNA sequence variation in the human genome. They are areas on the DNA structure where one to three nucleic acids may have a different sequence. For this type of mutation, there are usually only two possible sequences, or alleles.

Current SNP Scoring Methods—The most commonly used SNP sequencing methods are differential restriction and the Sanger method of sequencing. For differential restriction, the target is amplified by PCR, purified, cut with a restriction enzyme that only cuts one allele, and electrophoresed to visualize the fragment sizes (Liu, X-Y.; Nelson, D.; Grant, C.; Morthland, V.; Goodnight, S.; & Press, R. Diagn. Mol. Pathol. 1995, 4, 191-197). The Sanger method of sequencing involves a special PCR followed by slow electrophoresis on a long thin gel. These are labor-intensive methods. For large-scale sequencing, innovative methods have been developed using multi-well arrays. An example of a multi-well sequencing method is the “invader assay” that uses an enzyme to cut unpaired bases from double-stranded DNA formed by SNPs at a certain site. The cleaved piece then hybridizes to a signal probe where cleavage occurs again. This cleavage removes a fluorescent label from a quencher. The method requires one well for each allele and the aliquoting of different probes into each well. Amplification of signal occurs due to the recurrent enzymatic cleavage of supplied probes. The present invention overcomes the disadvantages associated with the currently used methods and does not require separate wells for each sequence of interest, does not require PCR, and does not require target labeling.

DNA Probe Arrays—DNA probe arrays enable large-scale diagnostic sequencing. Detection and scoring of a large number of mutations can be performed. PCR is needed to amplify and label the target nucleic acid before hybridization. Typically, DNA probes are immobilized on a silicon surface and incubated with multiple, labeled target nucleic acids. Hybrids are detected by the presence of labels at a probe site after washing away the non-bound solution components. Probe arrays are a massively parallel analysis technique, allowing the simultaneous detection of hundreds or even thousands of sequences (Meldrum, D. Genome Research. 2000, 10, 1288-1303). Although sequencing with these chips can be automated, it is not reliable for SNP sequencing unless a low complexity target sample is used. In order to differentiate single-base mismatches, short probes are required, but short sequences are often not unique in complex target DNA. Longer probes are unique but cannot differentiate between single-base mismatches. The present invention solves this problem by dual hybridization of target molecules to both a selection probe and a sequencing probe.

Peptide Nucleic Acid and Other Nucleic Acid Analogues—PNAs are homologues of DNA with a backbone polymer that contains aminoethyl glycine units to which the four bases [adenine (A), guanine (G), cytosine (C), and thymidine (T)] are attached. PNA hybridizes readily with complementary DNA and RNA sequences according to the same Watson-Crick binding rules as DNA/DNA hybrids. PNA is not charged so there is no need for high ionic strength during hybridization with DNA or RNA. The resulting PNA/DNA or PNA/RNA hybrid has a higher melting point than analogous DNA/DNA or RNA/RNA hybrids. Increased hybrid stability allows hybridization to be performed at higher temperatures, reducing hairpins and other interactions of complex, target nucleic acids that can interfere with diagnostic analysis. Single-base mismatches in target DNA sequences are extremely difficult to differentiate using DNA/DNA hybridization. Mismatches are more destabilizing to PNA/DNA hybrids (Egholm, M.; Buchardt, O.; Christensen, L.; Behrens, C.; Freier, S.; Driver, D.; Berg, R.; Kim, S.; Norden, B.; & Neilsen, P. Nature. 1993, 365, 566-568), however, allowing the determination of SNPs [Ross, P.; Lee, K.; & Belgrader, P. Anal. Chem., 1997, 69, 4197-4202; Jiang-Baucom, P.; Girard, P.; Butler, J.; & Belgrader, P. Anal Chem. 1997, 69, 4894-4898; Griffin, T.; Tang, W.; & Smith, L. Na.t Biotechnol. 1997, 15, 1368-1372). Unlike short DNA probes, PNA probes, once bound to long single-stranded DNA, are not displaced by complementary single-stranded DNA. Instead, a stable triplex is formed, leaving the probe in its original position. Any label attached to the PNA probe remains associated with the complementary target DNA.

Many labels are currently available for attachment to PNA. Fluorescent molecules are by far the most common label used to detect hybridization. Fluorescent microspheres contain many fluorescent molecules that can be present in several different ratios, resulting in many unique labels and sphere sizes. Microspheres can be visualized individually and counted using a microscope, allowing hybrid quantification. Other nucleic acid analogues have been synthesized that may be useful for SNP identification. Locked nucleic acids have been shown to discriminate single-base mismatches as well as PNA (Koshkin, et al., Tetrahedron 54 1998, 3607-3630). Other analogues include 2′-fluoro N3-P5′-phosphoramidates and 1′, 5′-anhydrohexitol nucleic acids (Schultz, D. & Gryaznov, S. Nucl, Acids Res. 1996, 24, 2966; Hendrix, C., et al., Chem. Eur. J. 1997, 3, 110).

Molecular Beacons—Molecular beacons are single-stranded DNA probes that are engineered to self-hybridize at the ends. A fluorescing molecule and a quenching molecule are held together during self-hybridization of the DNA. When the self-hybridization is destroyed by hybridization to a fully complementary nucleic acid, the fluorescent molecule's emitted light is no longer quenched and can be detected. Since the self-hybridized probe is highly stable and the length of the probes can be 20 or more nucleic acids long, they can be used for hybridization with complex nucleic acid samples. They are routinely used in PCR reactions with genomic DNA. Molecular beacons have been shown to differentiate SNPs well in diagnostic tests (Tapp, et al., Biotechniques 2000, 28,732-738).

Crosslinking Nucleic Acid Hybrids—Nucleic acids can be covalently bound using crosslinking agents. Psoralen can crosslink thymidines on two different nucleic acid strands (Pieles & Englisch. Nucl. Acids Res. 1989, 17, 285). Nucleotides with bromine or iodine in place of the 5-methyl group can crosslink to a nearby nucleic acid (Willis, et al., Science 1993, 262, 1255). For both psoralen and halogenated bases, the crosslinking occurs during excitation with certain wavelengths of light. Synthetic DNA oligomers can contain modified bases with unreacted crosslinking agents covalently bound or, in the case of psoralen, they can also intercalate between hybrid strands. Light activation then crosslinks the two strands together with a covalent bond that is more stable than hydrogen bonds.

### SUMMARY OF THE INVENTION

The present invention is drawn to a method of nucleic acid sequencing which comprises

hybridizing a selection probe with a conserved region of a target nucleic acid molecule that contains a sequence area of interest;

hybridizing a sequencing probe that is different from the selection probe to a variable region of the target nucleic acid molecule; and

determining the nucleic acid sequence of the area of interest only.

### BRIEF DESCRIPTION OF THE DRAWINGS

FIG. 1. Diagram of sample embodiment. Selection probes are immobilized to a surface. Complex, unlabeled target and sequencing probes are incubated with the selection probes forming dual-hybridized target strands. Detection of labels at individual selection probe sites determine the sequences of interest in the target sample.

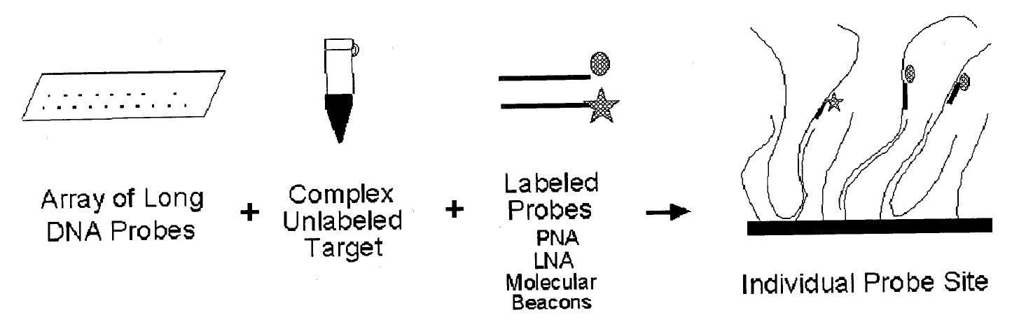

### DETAILED DESCRIPTION OF THE INVENTION

The present invention is drawn to a method for nucleic acid sequencing using the dual hybridization of a selection probe and a sequencing probe to a target nucleic acid. The selection probe is longer than the sequencing probe and is typically from 25 to 100 base pairs. The criterion for length is that the selection probe must be long enough to uniquely hybridize with nucleic acid segments in the target and the hybridization conditions for the selection probe and target must be compatible with the shorter PNA sequencing probe. It is also possible to ligate several selection probes end-to-end so that multiple probes exist in tandem on a single strand of nucleic acid. The selection probe is complementary to a sequence in the target near the sequence of interest. When the complementary sequence in the target hybridizes to the selection probe, it is immobilized, and the remainder of the target material can be removed by washing. This reduces the complex sample to only those segments of nucleic acid that contain the specific region of interest.

The sequencing probes are typically from 8 to 18 base pairs in length and hybridize to variable regions of the hybridized target and serve to identify the variable target sequence. The sequencing probe must be long enough to assure that the probability of a coincidental complementary sequence in the immobilized target is extremely low but it must be short enough to assure discrimination of a single-base mismatch in the hybrid it forms with the sequence of interest. In addition the hybridization conditions for the shorter PNA-DNA hybrid formed by the sequencing probe and target must be compatible with the hybridization conditions for the longer DNA-DNA hybrid formed by the selection probe and target. By using a sequencing probe, the need to label the target sample is eliminated.

The sequence of the steps can be variable, and include hybridization of the target sequence to the selection probe and sequencing probe and and hybrid detection. The hybridization of the sequencing probes and selection probes to the target are not order dependent and can occur simultaneously, separately and in any order.

The present invention provides a means for the rapid and efficient screening of a complex sample of nucleic acids for target sequences. For the screening of a sample for multiple sequences, the selection probes of approximately 50 bases in length may be immobilized onto a solid surface in an array. Suitable solid surfaces include, for example, glass, silica, gold or gold-coated surfaces, nylon, Teflon, or other polymers.

Each selection probe is complementary to a conserved region of a human gene where different alleles are known to exist. Other selection probes will be complementary to a highly conserved human sequence, such as the 18S ribosomal RNA, and will act as an internal, positive control. Sequencing probes will be composed of PNA or other synthetic DNA analog that forms hybrids with DNA that are more stable than normal DNA-DNA hybrids. Two different labels are needed for each area to be sequenced on a given segment of nucleic acid. Thus, if the same target strand has more than one area to be sequenced, more than two labels can be used. For example, blue, green, red, and yellow fluorescent microspheres can be used to sequence two areas that occur on the same target strand. Fluorescent microspheres can be attached to PNA using a biotin-streptavidin linkage. Other labels such as quantum dots, molecular beacons, mass labels, chemiluminescent or bioluminescent materials can also be used.

The selection probes will be approximately 50 bases long so that the selection probe/target hybrid is stable at the PNA/target hybridization temperature. The approximate melting temperature at 1 M Na+ for 50-mer DNA/DNA hybrids is above 70° C. The target DNA is sheered and heated to 100° C. in 6×SSC (90 mM trisodium citrate, pH 7.2/900 mM NaCl) to dissociate double-stranded target DNA into single-stranded target DNA. The target DNA is then applied to the surface of the DNA selection probe array and incubated at 50° C. After incubation, the array is washed with at 50° C. with 6×SSC. The labeled sequencing probes, which are approximately 12 bases in length, are then incubated with the array now containing the selection probe/target hybrids, in 3×SSC (45 mM trisodium citrate, pH 7.2/450 mM NaCl). After hybridization, the incubation solution is rinsed away and target/sequence probe/selection probe hybrids are detected. For example, a fluorescent microscope can be used to detect individual microspheres at the probe sites. If all four colors are bound to the probe site it means that both possible sequences at the two sites on the target are present. For example, target DNA from a person who is heterozygous at both sites would result in all four labels at the probe site. The ratios of labels that are present can offer more information about a target sample. For example, a target nucleic acid mix from a bacterial sample that may contain many strains could reveal the identities of the strains and their percentage of the total population. The use of large labels that can be seen individually and a flow-through cell that repeatedly cycle incubation solution across the array surface provides a means for analyzing the sample without the use of PCR.

As an optional variation to the method of the invention, crosslinking of the DNA selection probe to the target can be included. The selection probes would be designed for crosslinking to occur, such as by the inclusion of A-T base pairs near a psoralen modification. After incubation of target nucleic acid to the DNA array, the array is exposed to 350-nm light to form covalent bonds between the target nucleic acid and the thymidines of either or both DNA strands. This crosslinking step allows the PNA sequencing probe/target hybridization step to occur in extremely low salt conditions that would destroy DNA/DNA hybrids. The covalent bond, however, is not affected by the low ionic conditions and the target remains immobilized to the probe site.

By using a crosslinking step, labeling after hybridization can be performed. For example, PNA probes could have biotin and digoxigenin modifications. After hybridization, labeled avidin conjugates that bind to biotin and anti-digoxigenin antibody conjugates that bind to digoxigenin are applied. This label and detection system may be preferable under circumstances when, for example, the use of bulky labels such as fluorescent microspheres or enzymes is not desired. By using crosslinking of the nucleic acid and probe(s) it is also possible to omit, labeling of sequencing probes. For example, MALDI-TOF can be used to detect PNA probes of varying sizes that have been released from probe sites on a DNA array after matrix addition.

Since PNA/DNA and PNA/RNA hybrid melting temperatures vary little according to sodium concentration, a PNA sequencing probe/DNA target hybridization can be performed first to maximize SNP differentiation. In this case, a sequencing probe/target incubation in a low sodium buffer solution (i.e. 5 mM sodium phosphate, pH 7.0) is performed first. A vacuum or centrifugal filtration device can be used to remove the unbound sequencing probes from the much larger target fragments. The labels should be small enough allow easy size separation, therefore, fluorescent labels for example, can be used. The sequencing probe/target hybrids could then be applied to the array surface for hybridization with the previously immobilized selection probes. The sodium and buffer concentrations are increased to 6×SSC for optimal nucleic acid hybridization. After rinsing away unbound target, fluorescence detection for each type of fluorescent label used can be performed.

In addition to PNA, molecular beacons can be used as sequencing probes. Molecular beacons can be engineered so that only exact base pairing to a target is more stable than self-hybridization. Each probe is modified with a fluorophore and a quencher, so the target nucleic acids do not have to be labeled. The selection probes and sequencing probes are both DNA and highly stable so simultaneous hybridization can be used. Sheered, 100° C. target and sequencing probes would be applied to the DNA array in 6×SSC. After incubation, unbound probes and target are washed away and fluorescence detection is performed for each fluorophore type used.

We claim:

1. A method of nucleic acid sequencing which comprises
hybridizing a selection probe with a conserved region of a target nucleic acid molecule that contains a sequence area of interest;
hybridizing a sequencing probe that is different from the selection probe to a variable region of the target nucleic acid molecule; and
determining the nucleic acid sequence of the area of interest only.

2. The method of claim 1, wherein the selection probe is a DNA oligomer of approximately 25-100 nucleotides.

3. The method of claim 1, wherein one or more selection probes are immobilized to a surface in the form of an array before hybridization to the target nucleic acid.

4. The method of claim 1, wherein the selection probe is immobilized after hybridization with the target molecule.

5. The method of claim 1, further comprising ligating more than one selection probe together.

6. The method of claim 1, wherein the sequencing probe is approximately 8 to 18 base pairs.

7. The method of claim 1, wherein the sequencing probe is a synthetic nucleic acid that forms hybrids with DNA that are more stable than normal DNA-DNA hybrid.

8. The method of claim 7, wherein the sequencing probe is peptide nucleic acid.

9. The method of claim 1, wherein the sequencing probe contains locked nucleic acid(s).

10. The method of claim 1, wherein the sequencing probe contains a molecular beacon.

11. The method of claim 1, wherein the sequencing probe is labeled.

12. The method of claim 11, wherein the label on the sequencing probe is selected from the group consisting of fluorescent microspheres, quantum dots, molecular beacons, mass labels, chemiluminescent labels and bioluminescent labels.

13. The method of claim 1, which comprises more than one sequencing probe and wherein the sequencing probes are distinguishable each other by size or labels thereon.

14. The method of claim 1, further comprising crosslinking the selection probe to the target nucleic acid.

15. The method of claim 14, wherein the selection probe contains a psoralen modification and the selection probe and target nucleic acid are crosslinked by exposure to light of approximately 350 nm in wavelength.

16. The method of claim 14, further comprising labeling complexes of hybrids of target nucleic acid, selection probe and sequencing probe after hybridization is performed.

17. The method of claim 1, wherein the target molecule has one or more sequence areas of interest.

18. The method of claim 17, wherein the one or more sequence areas of interest is a single-nucleotide polymorphism.

19. The method of claim 1, wherein the selection probe hybridizes to allelic variants of a gene and the sequencing probes identify alleles in a target pool.

20. The method of claim 1, wherein the sequencing probe hybridizes only to completely complementary target nucleic acid sequences.

21. The method of claim 1, wherein recognition molecules are attached to the sequencing probes and wherein the recognition molecules interact with a labeling moiety.

22. The method of claim 21, wherein the labeling moiety is a streptavidin-conjugated microsphere and the recognition molecule is biotin.

23. The method of claim 21, wherein the labeling moiety is a anti-digoxigenin-conjugated microsphere and the recognition molecule is digoxigenin.
